(19) United States
(12) Patent Application Publication
(10) Pub. No.: US 2026/0341461 A1
(43) Pub. Date: Oct. 29, 2026

(54) SYSTEMS AND METHODS FOR VIRTUAL PEDIATRIC CRANIOFACIAL DEVELOPMENTAL ASSESSMENT AND STAGE-SPECIFIC INTERVENTION

(71) Applicant: Toothpillow, Inc., Draper, UT (US)

(72) Inventors: Chad Rasmussen, Draper, UT (US); Ryan Thomas Leary, Arvada, CO (US)

(21) Appl. No.: 19/662,182

(22) Filed: Apr. 29, 2026

Related U.S. Application Data
(60) Provisional application No. 63/796,637, filed on Apr. 29, 2025.

(51) Int. Cl. G16H 20/40; G16H 10/20; G16H 30/40

(52) U.S. Cl. CPC G16H 20/40; G16H 10/20; G16H 30/40

(57) ABSTRACT

A system for craniofacial developmental assessment and intervention planning includes a processor; and a memory coupled to the processor, the memory having instructions stored thereon which when executed by the processor, cause the system to: access a plurality of photographs associated with a patient; generate an oral image model based on the plurality of photographs; determine a level of deviation of the oral image model using a reference feature distribution; determine a craniofacial developmental assessment based on the level of deviation of the oral image model; and output an intervention plan correlated to the craniofacial developmental assessment.

### INCORPORATIONBYREFERENCE

This application claims the benefit of and priority to U.S. Provisional Patent Application No. 63/796,637 filed on Apr. 29, 2025, the entire content of which is incorporated by reference herein.

### TECHNICALFIELD

The subject matter of the present disclosure relates generally to systems and methods for virtual pediatric craniofacial developmental assessment and stage-specific intervention and, in particular, to remote assessment of craniofacial developmental findings using at-home photographs, questionnaire data, and calibrated image-based measurements.

### BACKGROUND

Proper craniofacial development during childhood can influence dental arch form, occlusal relationships, and other structural characteristics relevant to later oral and facial development. In pediatric patients, developmental stage can materially affect both what findings are clinically meaningful and what interventions are appropriate. Early identification of developmental issues, such as arch constriction, crossbite tendencies, disproportionate maxillomandibular relationships, or other craniofacial developmental deficiencies, may permit monitoring, behavioral intervention, myofunctional therapy, functional-appliance intervention, referral, or combinations thereof during a period in which growth-related change is still possible. Existing remote orthodontic and telehealth systems are generally directed to conventional tooth-movement treatment, adult or adolescent orthodontic workflows, or uncalibrated image review. Such systems typically do not provide a stage-aware pediatric assessment workflow based on at- home patient-captured photographs, calibrated dimensional analysis, symptom and history intake, and clinician-guided triage between virtual intervention and in-person referral. Systems lacking a dimensional reference in the captured images may also be limited in their ability to derive quantitative measurements suitable for consistent developmental assessment across time.

Accordingly, there is a need for systems and methods for virtual pediatric craniofacial developmental assessment and stage-specific intervention that can receive photographs captured in a remote environment, optionally with a calibration reference object of known dimensions, evaluate image-derived and questionnaire-derived information in view of a patient’s developmental stage, generate a craniofacial developmental assessment, and selectively route the patient to monitoring, therapy, functional-appliance intervention, re-assessment, and/or referral as appropriate.

### SUMMARY

An aspect of the present disclosure provides a system for craniofacial developmental assessment and intervention planning including: a processor; and a memory coupled to the processor, the memory having instructions stored thereon which when executed by the processor, cause the system to: access a plurality of photographs associated with a patient; generate an oral image model based on the plurality of photographs; determine a level of deviation of the oral image model using a reference feature distribution; determine a craniofacial developmental assessment based on the level of deviation of the oral image model; and output an intervention plan correlated to the craniofacial developmental assessment.

In an aspect of the present disclosure, the instructions, when executed by the processor, may further cause the system to: receive questionnaire data associated with the patient. The craniofacial developmental assessment may be further based on the questionnaire data.

In an aspect of the present disclosure, the questionnaire data may include symptom information, medical history information, dental history information, sleep-related information, feeding history information, mouth-breathing information, snoring information, habit information, and/or a combination thereof, and the system may be a system for virtual pediatric craniofacial developmental assessment and intervention planning.

In an aspect of the present disclosure, the instructions, when executed by the processor, may further cause the system to: determine a developmental stage of the patient based on an age of the patient, an eruption status visible in at least one of the plurality of photographs, or the questionnaire data.

In an aspect of the present disclosure, the craniofacial developmental assessment and the intervention plan may be further based on the developmental stage.

In an aspect of the present disclosure, at least one of the plurality of photographs may include a calibration reference object having markings of known spacing, and the instructions, when executed by the processor, may further cause the system to: derive at least one quantitative measurement from the plurality of photographs using the calibration reference object.

In an aspect of the present disclosure, the at least one quantitative measurement may include a dental dimensional measurement expressed in millimeters.

In an aspect of the present disclosure, the plurality of photographs may include photographs of an oral cavity of the patient taken from varying perspectives.

In an aspect of the present disclosure, the varying perspectives may include an anterior intraoral view, a maxillary occlusal view, a mandibular occlusal view, a buccal right view, a buccal left view, a frontal facial view, a lateral facial view, a tongue-elevated view, a closed-bite view, a smile view, and/or a combination thereof.

In an aspect of the present disclosure, the oral image model may include a compilation, stitched representation, panoramic representation, and/or three-dimensional representation of the plurality of photographs.

In an aspect of the present disclosure, the craniofacial developmental assessment may include developmental findings associated with dental arch form, crossbite, maxillomandibular proportion, arch constriction, crowding, palatal shape, or ankyloglossia.

In an aspect of the present disclosure, the instructions, when executed by the processor, may further cause the system to: determine whether the craniofacial developmental assessment falls within a predetermined scope eligible for virtual intervention; output the intervention plan when the craniofacial developmental assessment falls within the predetermined scope; and output a referral recommendation when the craniofacial developmental assessment falls outside the predetermined scope.

In an aspect of the present disclosure, the referral recommendation may identify at least one provider type.

In an aspect of the present disclosure, the instructions, when executed by the processor, may further cause the system to: retrieve explanatory content associated with the craniofacial developmental assessment or the intervention plan. The intervention plan may include a video, a downloadable file, a three-dimensional model, a prescription, a script, an exercise regimen, or explanatory multimedia content.

In an aspect of the present disclosure, the instructions, when executed by the processor, may further cause the system to: schedule a subsequent re-assessment based on the intervention plan, receive one or more additional photographs at the subsequent re-assessment, and update the craniofacial developmental assessment or the intervention plan based on the one or more additional photographs.

An aspect of the present disclosure provides a processor-implemented method for craniofacial developmental assessment and intervention planning, the method including: accessing a plurality of photographs associated with a patient; generating an oral image model based on the plurality of photographs; determining a level of deviation of the oral image model using a reference feature distribution; determining a craniofacial developmental assessment based on the level of deviation of the oral image model; and outputting an intervention plan correlated to the craniofacial developmental assessment.

In an aspect of the present disclosure, the method may further include: receiving questionnaire data associated with the patient. Determining the craniofacial developmental assessment may be further based on the questionnaire data. The method may be a method for virtual pediatric craniofacial developmental assessment and intervention planning.

In an aspect of the present disclosure, the method may further include: determining a developmental stage of the patient based on an age of the patient, an eruption status visible in at least one of the plurality of photographs, or questionnaire data.

In an aspect of the present disclosure, the method may further include: detecting a calibration reference object having markings of known spacing in at least one of the plurality of photographs; and deriving at least one quantitative measurement from the plurality of photographs using the calibration reference object.

In an aspect of the present disclosure, the method may further include: determining whether the craniofacial developmental assessment falls within a predetermined scope eligible for virtual intervention; outputting the intervention plan when the craniofacial developmental assessment falls within the predetermined scope; and outputting a referral recommendation when the craniofacial developmental assessment falls outside the predetermined scope.

Further details and aspects of exemplary embodiments of the present disclosure are described in more detail below with reference to the appended figures.

### BRIEFDESCRIPTIONOFTHEDRAWINGS

A better understanding of the features and advantages of the present disclosure will be obtained by reference to the following detailed description that sets forth illustrative embodiments, in which the principles of the present disclosure are utilized, and the accompanying drawings of which:

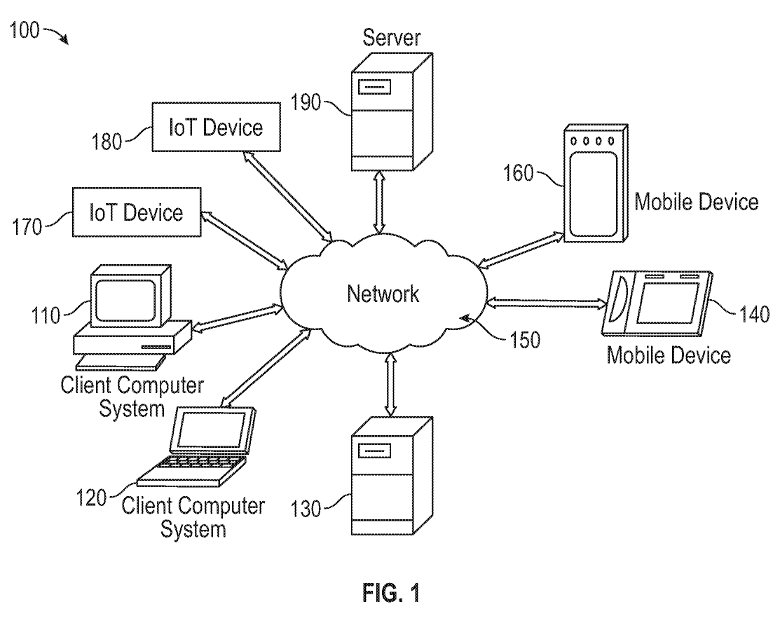

FIG. 1 is a diagram of a system for virtual pediatric craniofacial developmental assessment and intervention planning, in accordance with aspects of the present disclosure;

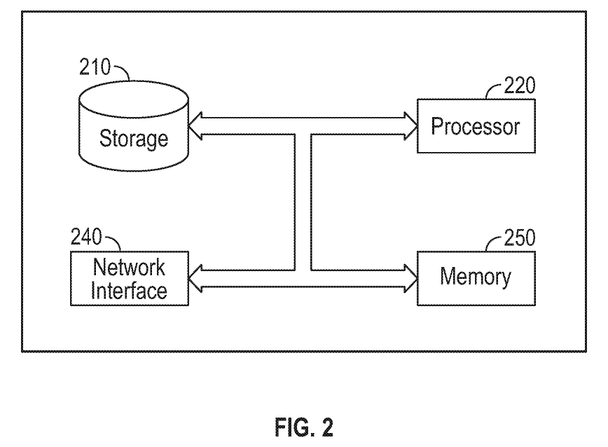

FIG. 2 is a block diagram of example components of a controller within the system of FIG. 1, in accordance with aspects of the present disclosure;

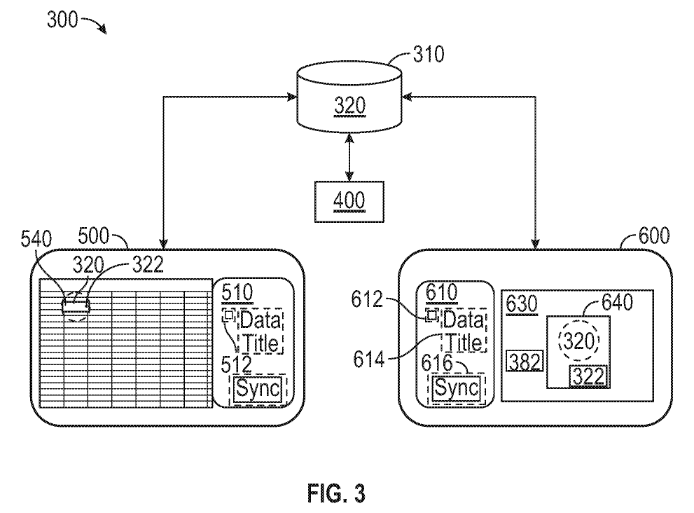

FIG. 3 is an illustration of a workflow of the system of FIG. 1, in accordance with aspects of the present disclosure;

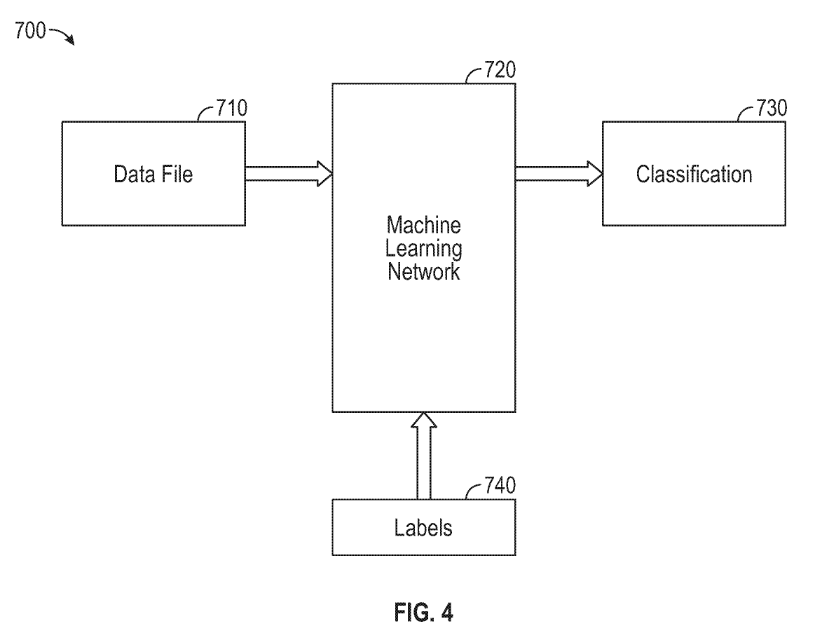

FIG. 4 is a block diagram of a machine learning network with inputs and outputs of a deep learning neural network of the system of FIG. 1, in accordance with aspects of the present disclosure;

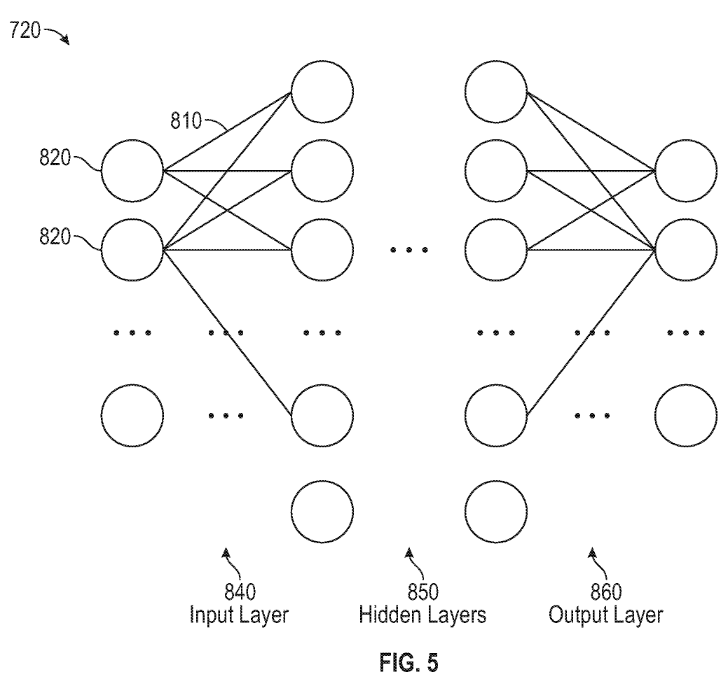

FIG. 5 is a diagram of the machine learning network with inputs and outputs of the deep learning neural network of the system of FIG. 1, in accordance with aspects of the present disclosure;

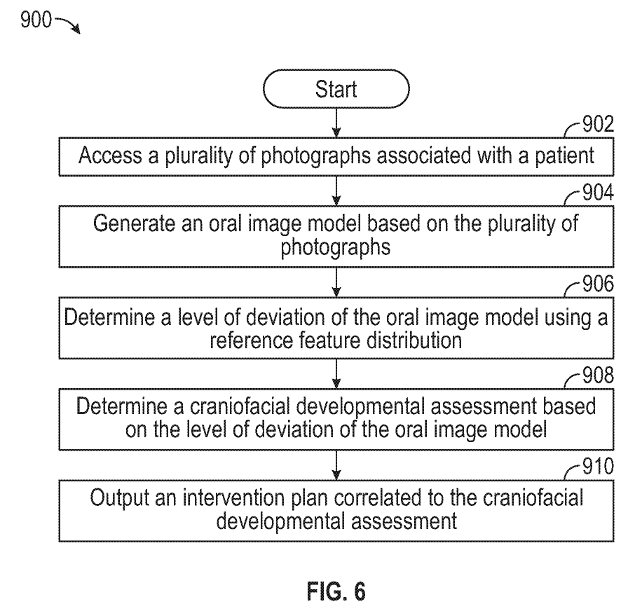

FIG. 6 is a flowchart of an exemplary method for virtual pediatric craniofacial developmental assessment and intervention planning, in accordance with aspects of the present disclosure;

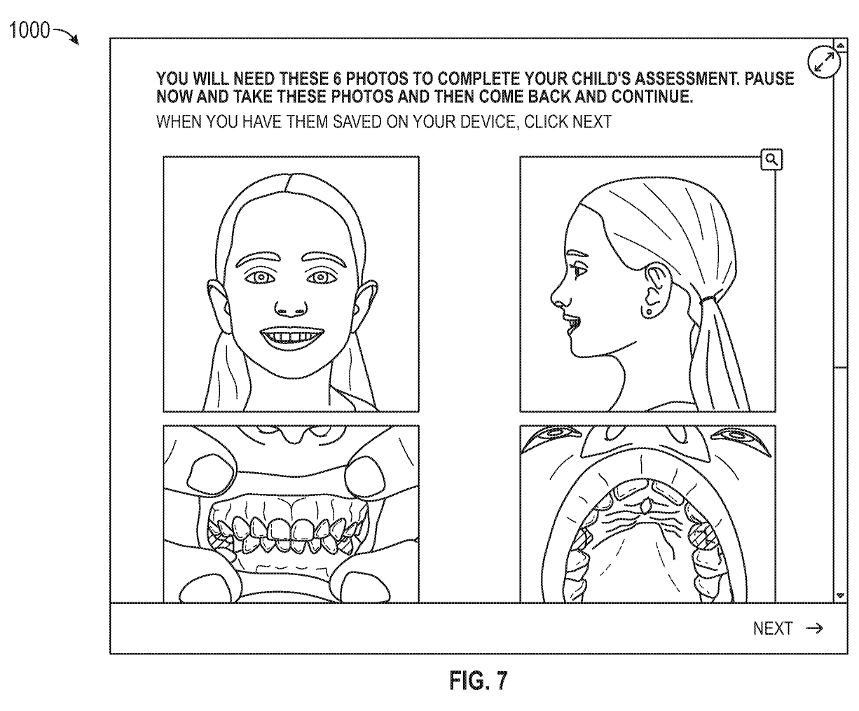

FIG. 7 is an exemplary questionnaire screen of the system of FIG. 1, in accordance with aspects of the present disclosure;

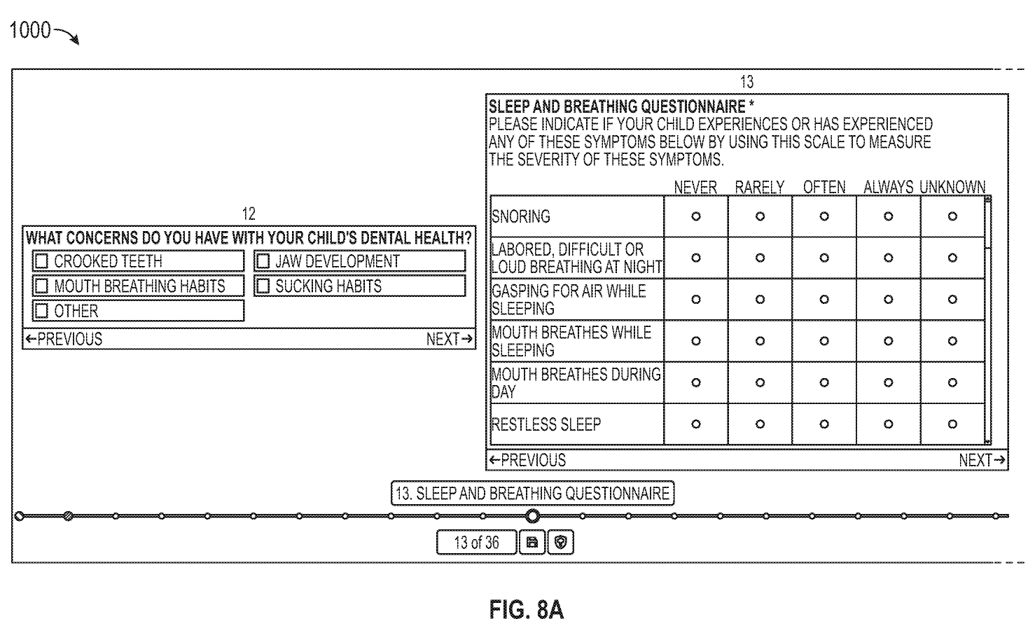

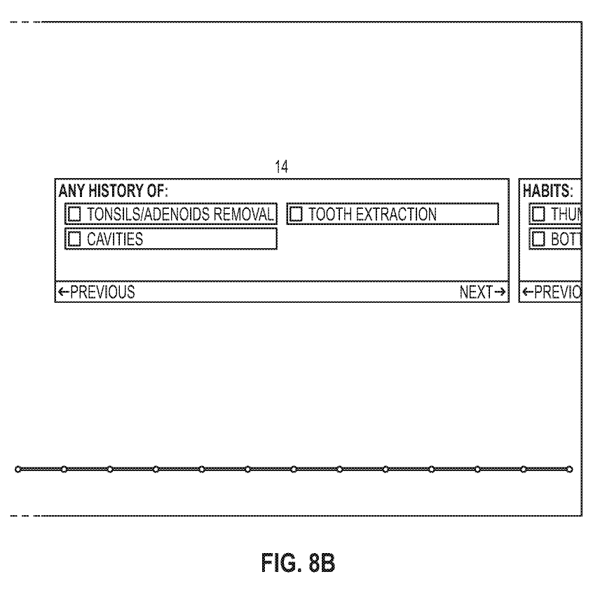

FIGS. 8A and 8B are an exemplary questionnaire screen of the system of FIG. 1, in accordance with aspects of the present disclosure; and

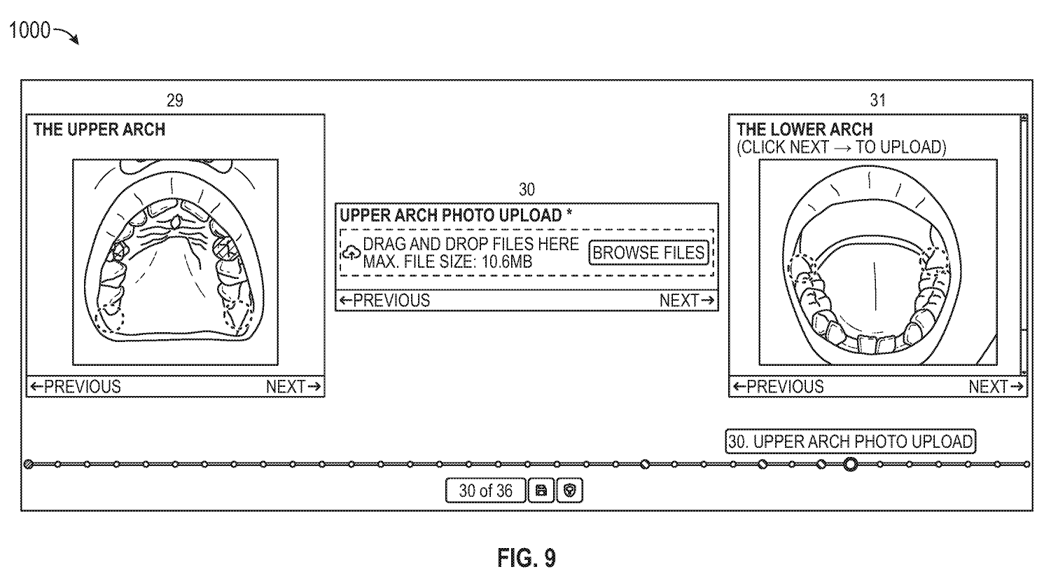

FIG. 9 is an exemplary questionnaire screen of the system of FIG. 1, in accordance with aspects of the present disclosure.

### DETAILEDDESCRIPTION

The present disclosure relates generally to systems and methods for virtual pediatric craniofacial developmental assessment and stage-specific intervention and, in particular, to remote evaluation of craniofacial developmental findings using patient photographs, questionnaire data, and image-based analysis.

Although the present disclosure will be described in terms of specific examples, it will be readily apparent to those skilled in this art that various modifications, rearrangements, and substitutions may be made without departing from the spirit of the present disclosure.

For the purpose of promoting an understanding of the principles of the present disclosure, reference will now be made to exemplary embodiments illustrated in the drawings, and specific language will be used to describe the same. It will nevertheless be understood that no limitation of the scope of the present disclosure is thereby intended. Any alterations and further modifications of the novel features illustrated herein, and any additional applications of the principles of the present disclosure as illustrated herein, which would occur to one skilled in the relevant art and having possession of this disclosure, are to be considered within the scope of the present disclosure.

As used herein, “data” refers to any form of information, values, content, or representational constructs that can be processed, transmitted, stored, and/or displayed by a computing system. Data may include numerical values, textual content, visual representations, graphical elements, metadata, or any other type of structured or unstructured information. For example, data may encompass the contents of a spreadsheet cell, including (e.g., timestamp of change, user details, or cell address) linked to a global identifier and updated to a central server. In another example, data may include information retrieved from a central server, which is linked to a shape in a presentation application, such as the contents of the spreadsheet cell and/or additional numerical, textual, and/or graphical parameters (e.g., xy coordinates related to shape, size, and/or position in a slide) related to the shape for synchronization.

As used herein, the term “synchronize” may generally refer to an ongoing process of synchronization using a central database, which causes a set of data and/or files to update and remain identical in one or more locations. In doing so, the locations may maintain consistent data to avoid conflicts. The system disclosed herein may use file synchronization in various locations, including one or more devices, applications, servers. etc. The synchronization process may implement version control, providing a synchronization solution for data and/or files that may be modified by multiple users in different locations. Therefore, the system may be configured to maintain separate file versions at different locations, which can each be updated and/or synchronized based on user input.

As used herein, a “shape” may generally refer to a geometric and/or freeform figure or object that can be created, manipulated, and/or displayed within a computer-based software application, such as a presentation program. A shape may include a text box, an image, a video, a flowchart, a table, a button, a comment, a basic geometric shape (e.g., lines, rectangles, squares, circles, ellipses, triangles, polygons, and/or stars), a freeform shapes (e.g., an irregular or user-defined contour and/or path that may be drawn using tools within an application), a complex shape (e.g., a combination and/or grouping of shapes to form compound figures or designs), and/or a decorative or illustrative shape (e.g., figures that incorporate embellishments and/or patterns for aesthetic or communicative purposes).

Ensuring proper craniofacial development, tongue posture, and arch formation from a young age is important because this period of growth offers an opportunity to guide development of oral and facial structures before they become less responsive to intervention later in life. During childhood, bones, muscles, and tissues are still developing, which can make it possible to identify and address oral and facial conditions that may influence long-term health. If these elements are not properly addressed early, habits such as mouth breathing, improper oral posture, or improper chewing may contribute to misaligned teeth, jaw issues, constricted arches, and other craniofacial developmental findings. Early assessment of these conditions may reduce the need for more extensive intervention later and may support improved oral function, facial development, and overall health over time.

Virtual consultations and intervention planning can allow patients and guardians to interact with a provider remotely, reducing the need for repeated in-person appointments. This may be particularly helpful for patients with limited access to specialty care, busy schedules, or geographic limitations. Remote visits can also provide greater flexibility in monitoring developmental changes, because patients and guardians can upload photographs or other information for review, allowing a provider to assess progress and recommend next steps over time. With these advantages, virtual care can provide a convenient, cost-effective, and efficient approach for managing craniofacial developmental assessment and intervention planning.

Integrating artificial intelligence into virtual consultations and intervention planning can offer significant advantages, particularly in analyzing patient photographs and related intake data. Artificial intelligence can improve the precision and speed of assessment by detecting and evaluating structural features from uploaded photographs, reducing the likelihood of human error and supporting more consistent assessments. Artificial-intelligence-driven tools can also track developmental changes over time, identify subtle shifts that might be missed in manual review, and assist in predicting likely trajectories or responses to intervention. These capabilities can improve the overall efficiency of remote assessment workflows and support more timely and tailored intervention planning. Accordingly, there is a need for systems and methods for virtual pediatric craniofacial developmental assessment and stage-specific intervention using patient photographs, questionnaire data, and image-based analysis.

Referring to FIG. 1, a system 100 for virtual pediatric craniofacial developmental assessment and intervention planning is shown in accordance with aspects of the present disclosure. The system 100 may include one or more client computer systems 110, 120, a cloud system 130, a network 150, one or more mobile devices 140, 160, one or more IOT devices 170, 180, and a server 190. The client computer systems 110, 120, the mobile devices 140, 160, and the IOT devices 170, 180 may communicate with the server 190 across the network 150. In some examples, the client computer systems 110, 120 may be used by clinicians, administrative personnel, or support personnel. In some examples, the mobile devices 140, 160 may be used by a patient, a parent, a guardian, or another caregiver to capture and submit photographs and questionnaire responses from a remote environment such as a home, school, daycare, or other non- clinical setting. The server 190 may be implemented as one server or as multiple servers distributed across one or more physical sites or cloud environments.

The system 100 may be configured to receive a plurality of photographs associated with a patient and to process those photographs together with questionnaire data to generate a craniofacial developmental assessment and an intervention-related output. In some embodiments, the photographs may include intraoral and extraoral photographs captured from multiple perspectives, such as an anterior intraoral view, a maxillary occlusal view, a mandibular occlusal view, a buccal right view, a buccal left view, a frontal facial view, a lateral facial view, a tongue-elevated view, a closed-bite view, a smile view, or combinations thereof. In some examples, at least one of the photographs may include a calibration reference object having markings of known spacing so that one or more quantitative measurements may be derived from the captured images. The photographs may be captured in one session or in multiple sessions and may be submitted in real time or asynchronously.

The server 190 may host or access software modules that perform one or more of photograph intake, image-quality assessment, calibration-object detection, landmark detection, segmentation, feature extraction, developmental-stage assessment, questionnaire processing, comparison to a reference feature distribution, intervention-plan generation, referral recommendation generation, explanatory-content retrieval, and follow-up scheduling. In some examples, the server 190 may receive photographs and questionnaire responses from a patient-facing application executed on one of the mobile devices 140, 160. In some examples, the server 190 may transmit assessment results, intervention content, reminders, or reassessment requests back to the mobile devices 140, 160 or to the client computer systems 110, 120 used by a clinician. The server 190 may further store or access patient-associated records that include photographs, questionnaire responses, age information, developmental-stage information, quantitative measurements, prior assessments, prior intervention plans, reassessment intervals, compliance-related information, and referral information.

In some embodiments, the server 190 may include or access an artificial-intelligence orchestration module configured to coordinate multiple processing operations associated with a patient case. The artificial-intelligence orchestration module may include one or more large language models, vision-language models, planning models, retrieval-augmented generation models, rules-based agents, task-specific machine-learning models, or combinations thereof. In some examples, the artificial-intelligence orchestration module may perform a multi-step workflow that includes evaluating submitted photographs, evaluating questionnaire responses, determining whether additional information or image recapture is needed, selecting one or more analysis modules, retrieving patient-specific or stage-specific reference information, generating a preliminary craniofacial developmental assessment, generating a proposed intervention plan, generating a proposed referral recommendation, generating explanatory content, and routing the patient case for clinician review, approval, modification, or escalation. In some embodiments, the artificial-intelligence orchestration module may autonomously select among available tools, models, content libraries, provider directories, reassessment protocols, and referral criteria based on patient-associated data and one or more confidence values, safety criteria, or scope-of-practice criteria.

The client computer systems 110, 120 may present one or more clinician-facing interfaces through which a provider may review submitted photographs, review extracted features, review measurements associated with a calibration reference object, review questionnaire responses, confirm or modify a craniofacial developmental assessment, review a proposed intervention plan, approve a referral recommendation, and monitor patient progress over time. In some examples, one of the client computer systems 110, 120 may be associated with a treating dentist, orthodontist, pediatric specialist, myofunctional therapist, care coordinator, or other provider. In some embodiments, one client computer system may be used for clinical review and another client computer system may be used for administrative workflow, scheduling, messaging, billing support, or content management. More than two client computer systems may be used within the system 100.

The mobile devices 140, 160 may each be any suitable user device, such as a smartphone, tablet, laptop computer, wearable device, or another portable computing device. In some examples, the mobile devices 140, 160 may execute a capture application that guides a user through photograph acquisition. The capture application may prompt the user to obtain particular views in a selected order, provide on-screen framing guidance, provide prompts related to head orientation, cheek retraction, mouth opening, or tongue elevation, and provide prompts for placement of a calibration reference object within the oral cavity or adjacent to dental structures visible in the image. The capture application may also present a questionnaire that collects symptom information, medical history information, dental history information, sleep-related information, feeding history information, mouth-breathing information, snoring information, habit information, and other patient-related data relevant to craniofacial developmental assessment and intervention planning.

In some embodiments, the cloud system 130 may provide elastic compute resources, model-serving infrastructure, distributed storage, audit logging, backup services, notification services, analytics infrastructure, or combinations thereof. The cloud system 130 may support one or more operations of the server 190, including large-scale image processing, machine-learning inference, longitudinal record management, and scheduled reassessment workflows. In some examples, the cloud system 130 may store reference datasets, reference feature distributions, developmental-stage criteria, intervention-plan libraries, explanatory-content libraries, provider directories, and reassessment protocols. In some examples, the cloud system 130 may support region-specific deployment, data partitioning, failover, and disaster-recovery operations.

The network 150 may be wired or wireless and may utilize technologies such as Wi-Fi, Ethernet, Internet Protocol, 4G, 5G, or other communication technologies. The network 150 may include, for example, a cellular network, residential broadband network, satellite communications network, private network, the Internet, local area network, wide area network, storage area network, campus area network, personal area network, metropolitan area network, or combinations thereof. The network 150 may support secure transmission of photographs, questionnaire data, assessment outputs, clinician notes, scheduling information, and other information exchanged among the illustrated components of the system 100.

The IOT devices 170, 180 may include peripheral or environmental devices that assist with patient interaction, data capture, communication, or monitoring. For example, an IOT device may include a camera accessory, a lighting accessory, a positioning aid, a calibration-reference reader, a kiosk component, a smart display, a biometric accessory, or another connected device used in connection with photograph capture or patient follow-up. In some embodiments, one or more of the IOT devices 170, 180 may be omitted. In other embodiments, additional IOT devices may be incorporated into the system 100.

The illustrated networked environment of system 100 is exemplary. Other systems, servers, storage resources, computing devices, software services, or interfaces not expressly illustrated in FIG. 1 may be included. One or more of the illustrated components may be omitted, consolidated, replicated, virtualized, or implemented in alternative form. Functions described as being performed by the server 190 may in some embodiments be performed in whole or in part by one or more client computer systems 110, 120, one or more mobile devices 140, 160, the cloud system 130, or combinations thereof.

Referring now to FIG. 2, there is shown a block diagram of example components of any of the systems, devices, or servers of FIG. 1 in accordance with aspects of the present disclosure. The components may include an electronic storage 210, a processor 220, a network interface 240, and a memory 250, among other components. The various components may be communicatively coupled with each other. The processor 220 may be any suitable processor, such as a single-core central processing unit, a multi-core central processing unit, a microprocessor, a microcontroller, a digital signal processor, a system-on-chip, an application-specific integrated circuit, a neural-processing unit, a graphics-processing unit, a tensor-processing unit, or another type of processor. In some embodiments, multiple processors may cooperate to perform photograph processing, quantitative measurement derivation, developmental-stage assessment, questionnaire interpretation, intervention-plan generation, or related functions.

The memory 250 may include volatile memory, non-volatile memory, or both. By way of example, the memory 250 may include RAM, flash memory, NAND flash memory, read-only memory, or other machine-readable media. The memory 250 may store processor-readable instructions that, when executed by the processor 220, cause the associated device or server to perform one or more operations described herein. Such operations may include receiving a plurality of photographs, generating an oral image model, detecting or interpreting a calibration reference object, determining a level of deviation using a reference feature distribution, generating a craniofacial developmental assessment, processing questionnaire data, determining a developmental stage, generating an intervention plan, generating a referral recommendation, retrieving explanatory content, scheduling a reassessment interval, receiving additional photographs at reassessment, and updating a prior assessment or plan.

The electronic storage 210 may include one or more storage devices used for storing photographs, patient-associated records, questionnaire responses, reference data, model parameters, quantitative measurements, developmental-stage criteria, clinician notes, reassessment schedules, intervention-plan templates, referral information, audit logs, and other information. The electronic storage 210 may include hard disk drives, solid-state drives, optical storage, network-attached storage, object storage, distributed storage, or other storage technologies. In some examples, the electronic storage 210 may store historical patient submissions so that the system may compare current photographs to prior photographs over time to identify trends, changes, progression, plateau, regression, or other longitudinal patterns.

The network interface 240 may support wired networking technologies, wireless networking technologies, or both. The network interface 240 may facilitate communication among the devices of FIG. 1 and may support encrypted transmission, authenticated sessions, device registration, access control, and data synchronization (e.g., patient-record synchronization). In some embodiments, the network interface 240 may receive image files, structured questionnaire responses, unstructured textual responses, scheduling data, clinician annotations, or notification data from remote devices and may transmit assessment results, reminders, multimedia content, and follow-up instructions back to those devices.

Additional components not expressly illustrated in FIG. 2 may also be included. For example, a device may include a display, a camera, a microphone, one or more additional sensors, one or more input devices, a secure enclave, or dedicated accelerators for image processing or machine-learning inference. A server implementation may include load balancers, hardware security modules, database interfaces, model-serving containers, message queues, or background job schedulers. The components shown in FIG. 2 are therefore examples, and systems, devices, and servers may include other components and may include multiple instances of the illustrated components.

Referring now to FIG. 3, there is shown an illustration of a workflow 300 of system 100 in accordance with aspects of the present disclosure. The workflow 300 generally may include a central server 310, data 320, a global identifier 322, a web application 400, a data management application 500, and a presentation application 600. In the present context, these elements may correspond to coordinated workflow components used for intake, processing, review, communication, synchronization, and follow-up within a virtual pediatric craniofacial developmental assessment platform. The workflow 300 is not limited to the particular applications, tools, or interfaces illustrated, and additional or alternative applications, tools, programs, and interfaces may be used.

In some embodiments, outputs, records, reports, or other information generated or managed by the system 100 may be provided through one or more output or productivity environments, including the web application 400, a mobile application, the data management application 500, a spreadsheet application, a document application, the presentation application 600, a clinical dashboard, a patient portal, an electronic health record interface, a provider portal, or combinations thereof. Accordingly, references herein to slides 610, 630, shapes 382, 616, 640, cells 510, 540, rows 512, panels 612, display regions 614, data fields, presentation elements, spreadsheet elements, interface elements, or other graphical or data objects may refer to structures used to display, organize, edit, export, synchronize, or otherwise manage patient-associated photographs, questionnaire responses, measurements, craniofacial developmental assessments, intervention plans, referral recommendations, explanatory content, reassessment schedules, compliance information, or other patient case data 320. Such objects may be used in clinician-facing reports, patient-facing summaries, downloadable files, educational presentations, structured case tables, longitudinal tracking interfaces, treatment-planning records, or other outputs associated with the virtual pediatric craniofacial developmental assessment and intervention workflow 300.

The central server 310 may be configured to store, update, and manage a state of data 320 using the global identifier 322, thereby creating a centralized repository for patient-related and workflow-related information. The central server 310 may include or host one or more databases configured to store, manage, track, and synchronize versions of data 320 across different sessions, devices, users, and stages of care. The data 320 may include photographs, questionnaire responses, age information, developmental-stage determinations, reference-feature comparisons, measurements derived from a calibration reference object, assessment outputs, intervention plans, referral recommendations, explanatory content associations, reassessment intervals, compliance-related information, and longitudinal progress information. In some examples, the global identifier 322 may associate related data records across different sessions, different devices, and different stages of care so that photographs, questionnaire submissions, clinician review records, intervention outputs, and reassessment data corresponding to a given patient or case may be retrieved and managed together. The central server 310 may communicate with various applications to handle data 320. For example, the central server 310 may manage and store data 320 received from the data management application 500 and data 320 displayed or otherwise utilized in the presentation application 600 by way of a shape 640. In another example, the central server 310 may display data 320 on the web application 400 to permit interaction with the data 320 and the workflow 300.

The central server 310 may protect data 320 using appropriate security and privacy controls. For example, the system may use authenticated access, fine-grained user permissions, role-based access controls, encryption in transit, encryption at rest, client-side encryption, audit logging, access tracking, secure token handling, temporary session credentials, checksums, or combinations thereof. Different classes of users may be granted different levels of access. For example, a guardian may be allowed to upload photographs and view educational materials, while a clinician may additionally review detailed measurements, modify an intervention plan, enter notes, or select a referral destination. An administrator may manage workflows, provider assignments, content libraries, and scheduling protocols. Security mechanisms may be adapted to regulatory requirements, institutional policies, and deployment-specific constraints.

The web application 400 may allow a user to interact with data 320, view the status of data 320, and send instructions to other in-application environments, such as the data management application 500 and the presentation application 600. In some examples, the web application 400 may provide one or more browser-accessible interfaces for a patient, guardian, provider, reviewer, or administrator. The web application 400 may present questionnaire forms, photograph-upload tools, appointment reminders, reassessment prompts, explanatory videos, downloadable materials, progress summaries, and messaging functions. The web application 400 may also present clinician-facing dashboards through which a provider may review image-derived findings, stage determinations, quantitative measurements, proposed plans, prior submissions, and patient-specific histories. In some examples, the web application 400 may include an overview of data 320 and other datasets used by the data management application 500 and the presentation application 600, such as a slide 630 or a shape 382 within a slide 630 in the presentation application 600. In other examples, the web application 400 may allow a user to create projects, case records, assessment sessions, reassessment events, or patient-specific workflows based on data 320. In still other examples, the web application 400 may allow a user to view, update, and manage data 320 based on a particular cell 540 in the data management application 500 and a particular shape 640 in the presentation application 600.

The data management application 500 may organize, normalize, validate, route, and synchronize incoming and outgoing data used by the system 100. In some embodiments, the data management application 500 may associate photograph files with a patient record, validate that required views have been submitted, verify completeness of questionnaire responses, assign a developmental-stage workflow, flag image-quality deficiencies, manage reassessment due dates, or route a case to a clinician queue. In some examples, the data management application 500 may include one or more data-entry or review elements such as a cell 510, a row 512, and a particular cell 540. The cell 510 may correspond to an individual data location within a structured patient-data environment. The row 512 may correspond to a collection of related data values associated with a particular patient, submission event, reassessment event, or workflow state. The particular cell 540 may correspond to a selected data field associated with a photograph, questionnaire response, developmental-stage value, quantitative measurement, intervention category, referral category, reassessment date, compliance indicator, or other case-specific value. In some examples, the data management application 500 may receive or generate structured outputs representing detected features, measurements, developmental-stage categories, confidence values, case-priority indicators, intervention categories, referral categories, or follow-up states. The data management application 500 may further maintain versioned histories of assessments and plans so that later reassessment results may be compared against earlier results.

The presentation application 600 may generate or display outputs that communicate assessment results and next-step recommendations. For example, the presentation application 600 may display an oral image model, a stitched image set, a panoramic view, a three-dimensional representation, extracted measurements, developmental findings, a recommended intervention pathway, a referral recommendation, a follow-up schedule, and explanatory multimedia content. In some examples, the presentation application 600 may present side-by-side comparisons of current and prior photographs, overlays indicating measured features or landmarks, and educational content tailored to the developmental finding or recommended intervention category. The presentation application 600 may be delivered through a browser, native application, tablet interface, clinician workstation, or another display environment. In some embodiments, the presentation application 600 may include one or more presentation elements such as a slide 630, a slide 610, a panel 612, a display region 614, a shape 616, and a shape 640. The slide 610 or slide 630 may correspond to an individual presentation page, screen, card, or visual output state used to communicate patient-specific information. The panel 612 may correspond to a subsection of the slide 610 or slide 630 or another interface area used to present a selected class of information, such as photographs, measurements, findings, intervention guidance, or reassessment timing. The display region 614 may correspond to a view area in which an oral image model, a patient photograph, a comparison image, a quantitative measurement overlay, or explanatory content is shown. The shape 616, shape 640, or shape 382 may correspond to a graphical element, annotation object, measurement overlay, highlight region, text box, button, icon, bounding region, or other display construct positioned within the presentation application 600 to identify, emphasize, or interact with information derived from data 320. In some examples, the slide 610, slide 630, panel 612, display region 614, shape 616, shape 640, and shape 382 may be dynamically updated based on a selected patient record, a selected reassessment period, a selected intervention output, or a selected data value from the data management application 500.

The workflow 300 may support longitudinal care and reassessment. For example, after an initial photograph submission and questionnaire intake, the central server 310 may assign a reassessment interval based on developmental stage, intervention type, clinician preference, or case complexity. At a later time, the system may prompt the user to submit additional photographs and updated questionnaire responses. The new information may be associated with the same global identifier 322 and compared to prior information stored in data 320. The system may then update the craniofacial developmental assessment, update an intervention plan, maintain the current plan, or generate a referral recommendation if a case has moved outside a virtual-intervention scope. In some examples, the workflow 300 may also support interim clinician reviews, messaging, progress tracking, synchronization of updated data values across the web application 400, the data management application 500, and the presentation application 600, and documentation of compliance or non-compliance with recommended activities.

Referring now to FIG. 4, a block diagram of a classification model 700 including a machine learning network 720 for evaluating patient-associated image and questionnaire data is shown in accordance with some aspects of the disclosure. In some systems, the machine learning network 720 may include a convolutional neural network, a regression model, a recurrent neural network, a transformer-based model, or another learned model. The machine learning network 720 may cooperate with one or more classification models 730, such as a convolutional neural network, decision tree, regression model, Naive Bayes model, k-nearest-neighbor model, ensemble model, or other classification or scoring model. In some examples, the classification model 700 may use a data file 710 and labels 740 for training, validation, or inference. The data file 710 may include patient photographs, segmented image regions, landmark coordinates, calibration-object information, questionnaire responses, developmental-stage labels, intervention labels, referral labels, or other structured or unstructured information relevant to craniofacial developmental assessment.

The machine learning network 720 may process one or more categories of inputs. For example, image inputs may include anterior intraoral views, maxillary occlusal views, mandibular occlusal views, buccal views, frontal facial views, lateral facial views, tongue-elevated views, or other patient photographs. Additional inputs may include age, symptom information, medical history information, feeding-history information, sleep-related information, snoring information, mouth-breathing information, habit information, and clinician-provided labels. In some examples, the model may generate one or more outputs associated with developmental stage, dental arch form, crossbite presence, maxillomandibular proportion, arch constriction, crowding, palatal-shape variation, ankyloglossia indicator, quantitative measurement refinement, intervention eligibility, intervention category, reassessment interval, or referral category.

In machine learning, a convolutional neural network is a class of artificial neural network that applies learned filters to localized portions of an input image to derive hierarchical features. In the present context, such features may correspond to dental contours, gingival margins, occlusal relationships, palatal boundaries, facial proportions, calibration markings, or other image-derived patterns relevant to craniofacial developmental assessment. A convolutional neural network may include convolution layers, activation layers, normalization layers, pooling layers, attention layers, skip connections, segmentation layers, or deconvolution layers. These operations may assist in extracting features from photographs that are later used alone or in combination with non-image inputs to generate assessments or recommendations.

Referring to FIG. 5, generally, the machine learning network 720 (FIG. 4) may include at least one input layer 840, a plurality of hidden layers 850, and at least one output layer 860. The input layer 840, the plurality of hidden layers 850, and the output layer 860 may each include neurons 820 or nodes. The neurons 820 between the various layers may be interconnected via weights 810. Each neuron 820 may compute an output value by applying a function to input values received from a preceding layer. The function applied to the input values may be determined by a vector of weights 810 and a bias. During training, learning may progress by iterative adjustment of the weights and biases based on training data and one or more loss functions. The learned weights and biases may correspond to filters or kernels associated with image regions, spatial patterns, questionnaire relationships, or other features useful for generating outputs from the system.

The output layer 860 may produce one or more logits, scores, classifications, rankings, regressions, segmentations, or confidence values. In some examples, the output layer 860 may produce a predicted developmental-stage category, a predicted presence or absence of a developmental finding, a refined dimensional measurement, a plan-selection score, a referral score, or a reassessment-timing recommendation. Although convolutional neural networks are used as examples, other machine-learning architectures may be used in place of or in combination with the illustrated arrangement.

The machine learning network 720 may be trained using labeled training data to optimize weights, biases, thresholds, and other learned parameters. For example, samples of patient-associated feature data may be labeled using clinician-reviewed classifications, clinician- assigned developmental stages, clinician-selected intervention categories, known measurement values, or later-confirmed follow-up outcomes. Training may include supervised learning, semi-supervised learning, self-supervised learning, transfer learning, active learning, federated learning, or combinations thereof. In some examples, the training data may include records accumulated through operation of the system across many patients and time points. In some examples, the training process may use separate training, validation, and test sets and may be periodically updated as additional labeled data becomes available.

In some embodiments, the training data, reference feature distribution, or both may be derived at least in part from accumulated patient-associated records collected through prior screening, assessment, monitoring, or intervention workflows. For example, the system may use a dataset including tens of thousands of screening records, such as about 40,000 or more patient screening records, and a longitudinal subset including thousands of patient records tracked over multiple years, such as about 13,000 or more longitudinal patient records collected over a period of at least three years. Such records may include patient photographs, questionnaire responses, age information, eruption-status information, clinician-reviewed developmental findings, intervention recommendations, referral outcomes, reassessment results, compliance information, and changes in craniofacial developmental features over time. The accumulated records may be used to generate, refine, stratify, or validate the reference feature distribution, developmental-stage criteria, deviation thresholds, intervention-plan selection criteria, referral criteria, machine-learning model parameters, or combinations thereof. In some examples, the records may be stratified by age, dentition stage, developmental stage, finding type, intervention pathway, reassessment interval, outcome, or other patient-associated variables so that the system evaluates a patient relative to a developmentally appropriate reference population.

Referring now to FIG. 6, a processor-implemented method 900 for virtual pediatric craniofacial developmental assessment and intervention planning is shown in accordance with aspects of the present disclosure. In some embodiments, the method 900 may be carried out by one or more processors associated with the system 100 described above, such as one or more processors of a server, cloud platform, client computer system, mobile device, or combinations thereof. The method 900 may be performed for a patient located in a remote patient capture environment and may use image data, questionnaire data, developmental-stage information, calibration-reference information, prior records, or combinations thereof to generate a craniofacial developmental assessment and an intervention-related output.

At block 902, the method 900 may include accessing a plurality of photographs associated with a patient. The plurality of photographs may include images captured by a patient, parent, guardian, caregiver, clinician, or other user using a smartphone, tablet, camera-equipped computer, or other image-capture device. In some examples, the photographs may include intraoral and extraoral photographs captured from different perspectives. Such perspectives may include, by way of example, an anterior intraoral view, a maxillary occlusal view, a mandibular occlusal view, a buccal right view, a buccal left view, a frontal facial view, a lateral facial view, a tongue-elevated view, a closed-bite view, a smile view, or other views suitable for evaluating craniofacial developmental features. Accessing the plurality of photographs may include receiving the photographs through a mobile application or web application, retrieving the photographs from electronic storage, associating the photographs with a patient-specific record, validating file integrity, validating that the photographs correspond to an expected capture session, determining whether a sufficient number of views has been received, determining whether one or more required views are missing, and performing preliminary image-quality review. In some embodiments, accessing the plurality of photographs may further include associating the photographs with metadata such as capture date, device type, patient identifier, case identifier, session identifier, upload source, or provider assignment.

In some embodiments, the photographs may be captured during a guided capture workflow in which an application prompts a user to obtain specific views in a prescribed order. The guided capture workflow may present instructions related to head orientation, camera distance, mouth opening, cheek retraction, tongue elevation, lighting, focus, field of view, or placement of a calibration reference object. In some examples, the method 900 may determine whether a submitted photograph satisfies one or more quality thresholds related to sharpness, illumination, framing, occlusion, motion blur, or visibility of relevant anatomy. When a photograph does not satisfy a threshold, the method 900 may prompt for recapture of that photograph or may flag the corresponding case for review.

At block 904, the method 900 may include generating an oral image model based on the plurality of photographs. The oral image model may include a compilation of the plurality of photographs, a stitched representation, a panoramic representation, a partial three-dimensional representation, a full three-dimensional representation, or another structured image representation derived from the submitted photographs. In some embodiments, generating the oral image model may include identifying common anatomical landmarks across multiple photographs, aligning the photographs relative to one another, compensating for differences in scale, orientation, exposure, or color balance, and combining image information from the plurality of photographs into a unified representation. In some examples, the method 900 may use image stitching, structure-from-motion techniques, multi-view reconstruction techniques, landmark-based registration, contour extraction, segmentation, or feature matching to generate the oral image model.

The oral image model may present a more comprehensive view of the oral cavity than any individual source photograph. For example, the oral image model may permit a processor or clinician to evaluate arch form, occlusal relationships, tooth positions, palatal geometry, visible soft-tissue relationships, or other image-derived features across multiple views in an integrated manner. In some embodiments, the oral image model may preserve links to the original source photographs so that derived findings or measured features may be traced back to one or more originating images. In some embodiments, the oral image model may be generated in multiple versions, such as a clinician-facing version with overlays, landmarks, or measurements and a patient-facing version with simplified presentation or explanatory annotations.

At block 906, the method 900 may include determining a level of deviation of the oral image model using a reference feature distribution. The reference feature distribution may correspond to a collection, library, model, statistical representation, learned distribution, or other reference dataset representing expected, typical, or target feature relationships for one or more patient populations, developmental stages, anatomical categories, or combinations thereof. In some embodiments, the reference feature distribution may be generated from previously collected records that include patient photographs, questionnaire responses, clinician-reviewed labels, developmental-stage labels, intervention outcomes, or combinations thereof. In some examples, the reference feature distribution may include image-derived features, quantitative measurements, shape descriptors, landmark relationships, arch-form descriptors, facial-proportion descriptors, or other features relevant to pediatric craniofacial developmental assessment.

In some embodiments, the reference feature distribution may be generated from accumulated patient-associated records, including screening records and longitudinal reassessment records collected over multiple years, that include patient photographs, questionnaire responses, clinician-reviewed labels, developmental-stage labels, intervention outcomes, or combinations thereof.

Determining the level of deviation may include extracting one or more features from the oral image model and comparing those features to one or more corresponding reference features represented in the reference feature distribution. For example, the method 900 may compare observed arch contours to reference arch-form patterns, compare observed occlusal relationships to expected relationships, compare measured or estimated widths, lengths, or ratios to reference distributions, compare facial-proportion indicators to age-appropriate or stage-appropriate distributions, or compare a combination of oral and facial features to one or more learned reference profiles. In some embodiments, the level of deviation may be expressed as a score, set of scores, classification, ranking, vector, probability, confidence value, severity value, threshold output, or other quantitative or qualitative representation. In some examples, the method 900 may calculate a global deviation value for the case as a whole and may also calculate local deviation values for particular structures, regions, or features.

Determining the level of deviation may further include applying one or more machine-learning models, rules-based models, or hybrid models. For example, a machine-learning model may process the oral image model to estimate feature distributions or severity values, while a rules-based module may interpret those values in view of stored criteria or thresholds. In some embodiments, a model may refine initial measurements or initial assessments by accounting for image-view quality, patient age, eruption status, arch asymmetry, tooth morphology, facial proportions, or other contextual variables. In some embodiments, the level of deviation may be derived with reference to a single generalized distribution or with reference to multiple distributions selected according to age, developmental stage, dentition stage, sex, treatment history, or other criteria.

At block 908, the method 900 may include determining a craniofacial developmental assessment based on the level of deviation of the oral image model. The craniofacial developmental assessment may include one or more developmental findings associated with a patient’s oral or facial structures. For example, the developmental assessment may include a finding associated with dental arch form, crossbite, maxillomandibular proportion, arch constriction, crowding, palatal shape, ankyloglossia indicator, facial growth tendency, or another craniofacial developmental feature. In some embodiments, determining the craniofacial developmental assessment may include mapping one or more deviation outputs to one or more developmental categories, generating a case summary, producing clinician-facing findings, assigning a severity value, or generating a case-specific explanation for use in downstream intervention planning.

In some examples, the craniofacial developmental assessment may be based not only on image-derived information, but also on contextual information associated with the patient. For example, the assessment may consider age, reported symptoms, feeding history, sleep-related information, mouth-breathing information, snoring information, habit information, developmental-stage information, prior assessments, prior measurements, prior plan adherence, or other information available to the system. In some embodiments, the craniofacial developmental assessment may include a single finding, multiple findings, or a composite profile of findings. In some embodiments, the craniofacial developmental assessment may include a machine-generated output that is subject to clinician review, confirmation, modification, supplementation, or override.

At block 910, the method 900 may include outputting an intervention plan correlated to the craniofacial developmental assessment. The intervention plan may correspond to one or more recommended next actions associated with the developmental findings determined at block 908. In some examples, the intervention plan may include a monitoring plan, a myofunctional-therapy-related plan, a functional-appliance-related plan, an exercise regimen, a follow-up schedule, a patient-education package, a provider review task, a reassessment request, or a combination thereof. Outputting the intervention plan may include storing the plan in association with the patient record, transmitting the plan to a patient-facing device, transmitting the plan to a clinician-facing device, presenting the plan within a web interface or mobile application, presenting educational content associated with the plan, scheduling reminders, or generating documentation for clinical review or recordkeeping.

In some embodiments, the intervention plan may include explanatory multimedia content selected based on the developmental assessment, patient age, developmental stage, intervention type, or other factors. For example, the intervention plan may include a video, downloadable file, three-dimensional model, prescription-related content, script, exercise instructions, or other explanatory content intended for a parent, guardian, patient, provider, or combinations thereof. In some embodiments, outputting the intervention plan may include generating different versions of the plan for different audiences, such as a simplified educational presentation for a guardian and a more detailed clinical view for a provider.

In embodiments, the method 900 may further include receiving questionnaire data associated with the patient. The questionnaire data may be received before, during, or after photograph capture and may be used together with the plurality of photographs in determining the craniofacial developmental assessment. The questionnaire data may include symptom information, medical history information, dental history information, sleep-related information, feeding history information, mouth-breathing information, snoring information, habit information, growth-related information, prior treatment information, compliance-related information, or other patient-associated information. In some examples, the questionnaire data may be collected through a structured intake form, branching questionnaire, interview workflow, clinician-completed form, or combination thereof. The method 900 may process the questionnaire data using a rules-based engine, a natural-language-processing workflow, a classification model, or another analysis routine so that the questionnaire data may contribute to case prioritization, developmental-stage assessment, intervention-path selection, or referral logic.

In embodiments, the method 900 may further include determining a developmental stage of the patient based on at least one of an age of the patient, an eruption status visible in at least one of the plurality of photographs, or questionnaire data. In some embodiments, determining the developmental stage may include identifying whether the patient is in a primary-dentition stage, an early mixed-dentition stage, a late mixed-dentition stage, a later dentition stage, or another stage relevant to evaluation and intervention planning. Eruption status may be inferred from visible presence, partial eruption, absence, or positional relationships of one or more teeth, including incisors, canines, molars, or other teeth. In some examples, the developmental stage may influence selection of the reference feature distribution used at block 906, interpretation of one or more deviation values, selection of intervention pathways, selection of reassessment intervals, or determination of whether the case falls inside or outside a scope eligible for virtual intervention.

In embodiments, the method 900 may further include detecting a calibration reference object having markings of known spacing in at least one of the plurality of photographs and deriving at least one quantitative measurement from the plurality of photographs using the calibration reference object. The calibration reference object may be a card, strip, appliance-associated component, ruler-like marker, fiducial object, printed target, or another object having markings separated by a known distance. In some examples, the calibration reference object may have markings at one-millimeter spacing or may include a known reference length such as 20 millimeters or 30 millimeters. Detecting the calibration reference object may include identifying the object in an image, segmenting the object, locating one or more markings, estimating object orientation, estimating scale, and calculating a pixel-to-distance conversion factor. Deriving the at least one quantitative measurement may include measuring a distance between selected dental landmarks, measuring a width, measuring an arch dimension, measuring a tooth-to-tooth relationship, measuring a palatal-related dimension, or deriving another dimension expressed in physical units such as millimeters. In some embodiments, the derived quantitative measurement may be used in generating the level of deviation at block 906, in determining the craniofacial developmental assessment at block 908, in selecting an intervention plan at block 910, or in comparing current and prior assessments over time.

In embodiments, the method 900 may further include determining whether the craniofacial developmental assessment falls within a predetermined scope eligible for virtual intervention, outputting the intervention plan when the craniofacial developmental assessment falls within the predetermined scope, and outputting a referral recommendation when the craniofacial developmental assessment falls outside the predetermined scope. The predetermined scope may be defined using one or more clinical, developmental, operational, or workflow-related criteria. For example, the predetermined scope may consider patient age, developmental stage, complexity of the detected findings, image quality, confidence values associated with the assessment, prior treatment history, availability of required views, presence of certain questionnaire responses, or other decision factors. When a case falls within the predetermined scope, the method 900 may provide an intervention plan suitable for remote monitoring, remote coaching, myofunctional exercises, functional-appliance guidance, or other virtual-care activities. When a case falls outside the predetermined scope, the method 900 may output a referral recommendation identifying a provider type, referral category, urgency level, or next-step instruction. The provider type may include, by way of example, an orthodontist, pediatric dentist, craniofacial specialist, myofunctional therapist, sleep-medicine specialist, primary-care provider, or another provider relevant to the case.

In some embodiments, the method 900 may further include scheduling a subsequent reassessment based on the intervention plan, the developmental stage, the case complexity, or a clinician determination. The reassessment may request one or more additional photographs, updated questionnaire responses, compliance-related information, or combinations thereof. Subsequent reassessment information may be associated with the same patient record and compared to prior information so that the method 900 may identify change over time. In some examples, a later execution of the method 900 may update a previously generated craniofacial developmental assessment, maintain a prior intervention plan, modify an intervention plan, or generate a referral recommendation based on progression, non-progression, plateau, regression, or other trends.

In some embodiments, one or more blocks of method 900 may be performed in a different order, repeated, omitted, subdivided, merged, or supplemented with additional operations. For example, questionnaire data may be received before block 902, calibration-object detection may occur during or after block 904, developmental-stage determination may occur before selection of the reference feature distribution at block 906, and referral logic may be applied after or together with output operations at block 910. One or more operations may be performed automatically by one or more processors, semi-automatically with clinician review, or manually with processor assistance.

In some embodiments, one or more operations of method 900 may be coordinated by an agentic artificial-intelligence workflow. The agentic artificial-intelligence workflow may decompose a patient case into a plurality of tasks, select one or more tools or models for performing the tasks, invoke the selected tools or models, evaluate intermediate outputs, determine whether additional photographs, questionnaire responses, clinician input, or prior patient records are needed, and generate a case-specific output based on the intermediate outputs. In some examples, the agentic artificial-intelligence workflow may use a large language model, a vision-language model, a retrieval-augmented generation process, a planner-executor architecture, a multi-agent architecture, or a combination thereof to reason over image-derived features, questionnaire-derived features, developmental-stage information, prior assessments, quantitative measurements, reference feature distributions, intervention criteria, referral criteria, and explanatory-content libraries. The agentic artificial-intelligence workflow may generate a preliminary assessment, recommended next action, proposed intervention plan, referral recommendation, reassessment schedule, patient-facing explanation, clinician-facing summary, or combination thereof, subject to clinician review, automated safety checks, confidence thresholds, or predetermined scope criteria.

The method 900 may be implemented using one or more machine-learning models, one or more rules-based models, or a hybrid decision framework. For example, a machine-learning model may extract image features from the plurality of photographs, estimate measurements, classify developmental-stage indicators, generate preliminary developmental findings, or score intervention eligibility. A rules-based layer may then apply stored thresholds, plan-selection criteria, reassessment criteria, or referral criteria. In other embodiments, the method 900 may use an end-to-end learned model to generate one or more of the level of deviation, the craniofacial developmental assessment, the intervention plan, or the referral recommendation. Various combinations of model architectures may be used, including convolutional neural networks, recurrent neural networks, transformer-based models, regression models, decision trees, ensemble models, or other computational models.

The method 900 may further support patient education and provider communication. For example, after outputting an intervention plan or referral recommendation, the method 900 may retrieve explanatory content associated with the developmental findings, selected plan category, or patient developmental stage. The explanatory content may include text, graphics, video, interactive models, guided instructions, reminders, progress summaries, or provider-authored notes. In some examples, the content may be selected from a content library indexed according to developmental finding, developmental stage, intervention category, provider preference, or other factors. The resulting output may therefore provide not only a plan or referral pathway, but also materials that facilitate understanding, compliance, and follow-up.

The method 900 shown in FIG. 6 is therefore an example of a processor-implemented workflow that uses patient photographs and related information to generate a craniofacial developmental assessment and to support stage-appropriate intervention planning, remote follow-up, and referral decision-making.

Referring to FIGS. 7-9, an example user portal interface 1000 is shown in accordance with aspects of the present disclosure. The user portal interface 1000 may be presented on one or more mobile devices 140, 160, on one or more client computer systems 110, 120, or through a web application 400 accessible through a browser or native application environment. The user portal interface 1000 may guide a patient, parent, guardian, caregiver, clinician, or other user through capture of photographs, submission of questionnaire information, review of instructions, and communication of information to the server 190 through the network 150. In some embodiments, the user portal interface 1000 may form part of a patient-facing capture application that assists with collection of the photograph set and intake information used for craniofacial developmental assessment and intervention planning. The user portal interface 1000 may present step-by-step instructions, visual examples, reminders, completion-status indicators, upload-confirmation indicators, retake prompts, and educational content so that a user may more reliably provide information suitable for subsequent processor-based analysis.

Referring particularly to FIG. 7, the user portal interface 1000 may present one or more photograph-capture screens through which a user is instructed to obtain a plurality of photographs associated with a patient. In some examples, the user portal interface 1000 may display thumbnail examples, outlines, overlays, framing guides, checklists, or textual prompts corresponding to desired image views. The desired image views may include one or more intraoral and extraoral views, such as an anterior intraoral view, a maxillary occlusal view, a mandibular occlusal view, a buccal right view, a buccal left view, a frontal facial view, a lateral facial view, a tongue-elevated view, a closed-bite view, a smile view, or combinations thereof. In some embodiments, the user portal interface 1000 may instruct the user regarding lighting, camera angle, cheek retraction, tongue position, head orientation, mouth opening, and image focus so that anatomical structures relevant to developmental assessment are visible. The user portal interface 1000 may also indicate whether a view is required, optional, incomplete, accepted, or should be retaken.

In some embodiments, the photograph-capture screen of FIG. 7 may be arranged to support remote collection of a photograph set from a home or other remote patient capture environment. The user portal interface 1000 may provide prompts directing a parent or guardian to stand at a recommended distance from the patient, use a flash or auxiliary light source, stabilize the mobile device, rotate the camera to a desired orientation, or repeat a capture if blur or poor framing is detected. In some examples, the user portal interface 1000 may present one view at a time in a prescribed sequence so that the capture session progresses through a standardized workflow. In other examples, the user portal interface 1000 may display a multi-item checklist showing all requested views and the completion status of each view. The user portal interface 1000 may also store partially completed capture sessions so that a user may resume the workflow later.

In some embodiments, the user portal interface 1000 may present instructions associated with a calibration reference object when quantitative measurements are to be derived from one or more photographs. For example, the user portal interface 1000 may instruct the user to place a calibration reference object having markings of known spacing within the oral cavity or adjacent to dental structures visible in a selected photograph. The user portal interface 1000 may display an example image showing proper placement of the calibration reference object, acceptable orientation of the markings, or acceptable visibility of the reference object relative to teeth, gingiva, or palatal structures. In some examples, the user portal interface 1000 may provide a warning if the calibration reference object is not sufficiently visible or if a submitted image is not suitable for measurement derivation.

The photographs collected through the user portal interface 1000 may be transmitted to the server 190 and associated with a patient record, session identifier, or other case identifier. In some embodiments, the user portal interface 1000 may automatically compress, encrypt, tag, or timestamp images before transmission. The server 190 may then access the photographs to generate an oral image model, determine a level of deviation using a reference feature distribution, determine a craniofacial developmental assessment, and generate an intervention-related output as described elsewhere herein. In some examples, the user portal interface 1000 may subsequently display status information indicating that the photograph set has been received, is under review, requires additional images, or has advanced to a next step in the assessment workflow.

Referring now to FIGS. 8A, 8B, and 9, the user portal interface 1000 may further include questionnaire screens through which questionnaire data associated with the patient is collected. The questionnaire screens may be completed before photograph capture, during photograph capture, after photograph capture, or at one or more later reassessment times. In some embodiments, the questionnaire screens may gather symptom information, medical history information, dental history information, feeding history information, sleep-related information, mouth-breathing information, snoring information, habit information, prior treatment information, developmental information, compliance-related information, or other information relevant to craniofacial developmental assessment and intervention planning. The user portal interface 1000 may present the questionnaire as a sequence of individual prompts, grouped sections, branching forms, selectable response options, free-text fields, sliders, checkboxes, drop-down menus, or combinations thereof.

In some examples, the exemplary questionnaire screens shown in FIGS. 8A, 8B, and 9 may prompt a user for information such as whether the patient snores, mouth breathes, has feeding difficulties, has sleep-related concerns, has known dental crowding, has crossbite history, has oral habits, has received prior orthodontic or therapeutic treatment, or has symptoms that may affect a provider’s assessment of developmental stage or intervention suitability. The user portal interface 1000 may also request age information, date-of-birth information, caregiver observations, eruption-related observations, or prior provider recommendations. In some embodiments, one or more questionnaire responses may affect branching logic so that later prompts are selected based on earlier answers. For example, if a user indicates that the patient has previously received care, the user portal interface 1000 may present follow-up prompts requesting the type of care, timing of care, or reasons for discontinuation. If a user indicates concern regarding breathing or sleep, the user portal interface 1000 may present additional prompts directed to frequency, duration, severity, prior evaluation, or provider involvement.

The questionnaire data collected through the user portal interface 1000 may be processed together with the plurality of photographs so that the craniofacial developmental assessment is based on both image-derived and questionnaire-derived information. In some embodiments, the questionnaire data may assist in determining a developmental stage of the patient, selecting a reference feature distribution, prioritizing case review, selecting an intervention pathway, or determining whether a case falls within a predetermined scope eligible for virtual intervention. In some examples, questionnaire responses may be interpreted using a rules-based engine, a scoring model, a natural-language-processing routine for free-text responses, or another processor-based workflow. The resulting information may then be stored in association with the patient record and presented to a clinician through one or more clinician-facing interfaces.

In some embodiments, the user portal interface 1000 may support age-related or stage-related intake logic. For example, the user portal interface 1000 may classify or sort submitted information according to one or more age groupings, such as 0-36 months, 3-12 years old, 13-17 years old, or 18 years old and older. In other embodiments more closely aligned with pediatric developmental assessment, the user portal interface 1000 may support sorting or evaluation based on dentition stage, eruption status, or another developmental-stage framework. Such information may be used by the server 190 in determining whether a case is appropriate for a particular intervention pathway, whether additional information should be requested, whether reassessment should occur at a particular interval, or whether referral should be recommended.

The user portal interface 1000 may further support communication of intervention-related outputs back to the user after the system has completed at least a portion of the assessment workflow. In some examples, the user portal interface 1000 may display an intervention plan, a monitoring recommendation, a myofunctional-therapy-related recommendation, a functional-appliance-related recommendation, a reassessment schedule, or a referral recommendation. The user portal interface 1000 may also present explanatory content associated with the developmental assessment or intervention plan, such as videos, downloadable files, exercise instructions, progress information, or other educational materials. In some embodiments, the user portal interface 1000 may present content differently depending on whether the viewer is a parent, guardian, patient, or provider.

In some embodiments, the user portal interface 1000 may support longitudinal follow-up after an initial submission. For example, the user portal interface 1000 may issue reminders for submission of additional photographs, updated questionnaire responses, compliance-related updates, or scheduled reassessments. The user portal interface 1000 may display upcoming due dates, prior submissions, completion percentages, progress summaries, or alerts indicating that a reassessment is due. The user portal interface 1000 may also permit a user to report intervening events, changes in symptoms, changes in treatment compliance, or questions for provider review. Information obtained through such follow-up interactions may be transmitted to the server 190 and associated with a prior patient record so that later assessments may be compared against earlier assessments over time.

The user portal interfaces 1000 shown in FIGS. 7-9 are examples. Additional screens, prompts, widgets, menus, capture overlays, educational displays, messaging interfaces, scheduling interfaces, consent forms, or upload workflows may also be used. Likewise, one or more of the illustrated user portal interfaces 1000 may be omitted, combined, reordered, or adapted for different device types, patient populations, care pathways, or deployment environments.

Certain embodiments of the present disclosure may include some, all, or none of the above advantages and/or one or more other advantages readily apparent to those skilled in the art from the drawings, descriptions, and claims included herein. Moreover, while specific advantages have been enumerated above, the various embodiments of the present disclosure may include all, some, or none of the enumerated advantages and/or other advantages not specifically enumerated above.

The embodiments disclosed herein are examples of the disclosure and may be embodied in various forms. For instance, although certain embodiments herein are described as separate embodiments, each of the embodiments herein may be combined with one or more of the other embodiments herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

The phrases “in an embodiment,” “in embodiments,” “in various embodiments,” “in some embodiments,” or “in other embodiments” may each refer to one or more of the same or different example embodiments provided in the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

What is claimed is:

1. A system for craniofacial developmental assessment and intervention planning, comprising: a processor; and
a memory coupled to the processor, the memory having instructions stored thereon which when executed by the processor, cause the system to:
access a plurality of photographs associated with a patient;
generate an oral image model based on the plurality of photographs;
determine a level of deviation of the oral image model using a reference feature
distribution;
determine a craniofacial developmental assessment based on the level of deviation of the oral image model; and
output an intervention plan correlated to the craniofacial developmental
assessment.

2. The system of claim 1, wherein the instructions, when executed by the processor, further cause the system to:
receive questionnaire data associated with the patient,
wherein the craniofacial developmental assessment is further based on the questionnaire data.

3. The system of claim 2, wherein the questionnaire data includes symptom information, medical history information, dental history information, sleep-related information, feeding history
information, mouth-breathing information, snoring information, habit information, or a combination thereof, and
wherein the system is a system for virtual pediatric craniofacial developmental assessment and intervention planning.

4. The system of claim 2, wherein the instructions, when executed by the processor, further cause the system to:
determine a developmental stage of the patient based on at least one of an age of the patient, an eruption status visible in at least one of the plurality of photographs, or the questionnaire data.

5. The system of claim 4, wherein the craniofacial developmental assessment and the intervention plan are further based on the developmental stage.

6. The system of claim 1, wherein at least one of the plurality of photographs includes a calibration reference object having markings of known spacing, and
wherein the instructions, when executed by the processor, further cause the system to: derive at least one quantitative measurement from the plurality of photographs
using the calibration reference object.

7. The system of claim 6, wherein the at least one quantitative measurement includes a dental dimensional measurement expressed in millimeters.

8. The system of claim 1, wherein the plurality of photographs include photographs of an oral cavity of the patient taken from varying perspectives.

9. The system of claim 8, wherein the varying perspectives include an anterior intraoral view, a maxillary occlusal view, a mandibular occlusal view, a buccal right view, a buccal left view, a frontal facial view, a lateral facial view, a tongue-elevated view, a closed-bite view, a smile view, or a combination thereof.

10. The system of claim 1, wherein the oral image model includes a compilation, stitched representation, panoramic representation, or three-dimensional representation of the plurality of photographs.

11. The system of claim 1, wherein the craniofacial developmental assessment includes at least one developmental finding associated with at least one of dental arch form, crossbite, maxillomandibular proportion, arch constriction, crowding, palatal shape, or ankyloglossia.

12. The system of claim 1, wherein the instructions, when executed by the processor, further cause the system to:
determine whether the craniofacial developmental assessment falls within a predetermined scope eligible for virtual intervention;
output the intervention plan when the craniofacial developmental assessment falls within the predetermined scope; and
output a referral recommendation when the craniofacial developmental assessment falls outside the predetermined scope.

13. The system of claim 12, wherein the referral recommendation identifies at least one provider type.

14. The system of claim 1, wherein the instructions, when executed by the processor, further cause the system to:
retrieve explanatory content associated with the craniofacial developmental assessment or the intervention plan,
wherein the intervention plan includes at least one of a video, a downloadable file, a three-dimensional model, a prescription, a script, an exercise regimen, or explanatory multimedia content.

15. The system of claim 1, wherein the instructions, when executed by the processor, further cause the system to:
schedule a subsequent re-assessment based on the intervention plan,
receive one or more additional photographs at the subsequent re-assessment, and
update the craniofacial developmental assessment or the intervention plan based on the one or more additional photographs.

16. A processor-implemented method for craniofacial developmental assessment and intervention planning, the method comprising:
accessing a plurality of photographs associated with a patient; generating an oral image model based on the plurality of photographs;
determining a level of deviation of the oral image model using a reference feature distribution;
determining a craniofacial developmental assessment based on the level of deviation of the oral image model; and
outputting an intervention plan correlated to the craniofacial developmental assessment.

17. The method of claim 16, further comprising:
receiving questionnaire data associated with the patient,
wherein determining the craniofacial developmental assessment is based further on the questionnaire data, and
wherein the method is a method for virtual pediatric craniofacial developmental assessment and intervention planning.

18. The method of claim 16, further comprising:
determining a developmental stage of the patient based on at least one of an age of the patient, an eruption status visible in at least one of the plurality of photographs, or questionnaire data.

19. The method of claim 16, further comprising:
detecting a calibration reference object having markings of known spacing in at least one of the plurality of photographs; and
deriving at least one quantitative measurement from the plurality of photographs using the calibration reference object.

20. The method of claim 16, further comprising:
determining whether the craniofacial developmental assessment falls within a predetermined scope eligible for virtual intervention;
outputting the intervention plan when the craniofacial developmental assessment falls within the predetermined scope; and
outputting a referral recommendation when the craniofacial developmental assessment falls outside the predetermined scope.
